(19) United States
(12) Patent Application Publication
(10) Pub. No.: US 2025/0136537 A1
(43) Pub. Date: May 1, 2025

(54) SYSTEM AND PROCESS FOR CONTINUOUS PREPARATION OF meta-CRESOL BY NOx

(71) Applicant: DEEPAK NITRITE LIMITED, Vadodara (IN)

(72) Inventors: Aminmahamad N. ISMAILI, Vadodara (IN); Vilas PATIL, Vadodara (IN); Javedhusen K MANSURI, Vadodara (IN)

(21) Appl. No.: 18/694,208

(22) PCT Filed: May 18, 2022
(86) PCT No.: PCT/IB2022/054593

§ 371 Date: Mar. 21, 2024

(30) Foreign Application Priority Data
Sep. 24, 2021 (IN) 202121043384

(51) Int. Cl. C07C 37/045; B01D 19/00; B01J 19/00; B01J 19/18; B01J 19/30; C01B 21/34

(52) U.S. Cl. CPC C07C 37/045; B01D 19/0005; B01J 19/0013; B01J 19/0066; B01J 19/1862; B01J 19/30; C01B 21/34; B01J 2219/00033; B01J 2219/00103

(57) ABSTRACT

A system and process for continuous preparation of meta-cresol. The system may include one or more components such as a gas mixing unit, a reactor unit connected to a coiled reactor, a diazotization unit including a trickle bed reactor unit having a set of packed columns, an excess NOx removal unit, a hydrolysis reactor unit, a layer separation unit, a spent acid recovery unit, and a meta-cresol collection unit.

### TECHNICAL FIELD

The present disclosure described herein, in general, relates to a system and process for preparation of m-cresol. More particularly, the present disclosure relates to continuous process for development of m-cresol through NOx route.

### BACKGROUND

The compound meta-Cresol (CH₃·C₆H₄·OH) is an organic compound, having IUPAC name 3-methyl phenol.

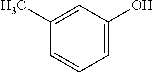

meta-Cresol is a precursor to numerous compounds such as fenitrothion and fenthion like pesticides, synthetic vitamin E by methylation to give the 2,3,6-trimethylphenol, and amyl meta-cresol like antiseptics. In the industrial field, different standards, and specifications of m-Cresol in view of purity and quality are required.

Conventionally, meta-cresol is obtained by de-butylation of tertiary butylated m-cresol by following a distillation of butylated m-, p-cresol mixtures. In some cases, meta-cresol is obtained by using zeolite/urea-based separation processes applied on cresol mixtures. Another, relatively old route of preparation of m-cresol involves Sandmeyer diazotization process using m-toluidine as a raw material. Meta-cresol is traditionally extracted from coal tar, the volatile materials obtained in the production of coke from (bituminous) coal.

In state of the art, Chinese Application No. 101125800 discloses about a process consisting of reacting sodium nitrite solution with meta-toluidine sulfate salt. The major drawback of the process was removal of unreacted sodium nitrite from a diazotization chamber. The inability to remove unreacted sodium nitrite leads to formation of a tar like impurity matter which is difficult to process further or to treat as an effluent at a large-scale production of meta-cresol. Further, the reaction between solid-liquid components causes the formation of by-products and impurities at the diazotization stage.

In state of the art, Chinese Application No. 101402552 discloses about direct hydrolysis of meta-aminotoluene vitriol in presence of aqueous ammonium chloride solution excluding incorporation of sodium nitrite. The said process does not provide any solution for recycling of effluents.

Attempts have been made to substitute sodium nitrite with NOx route for m-cresol production by reacting nitrogen oxide (NO) with m-Toluidine. However, this process at larger extent is difficult to scale up, handling, and results into lower yield, more tar formation, and scaling of reactor. The diazonium salt obtained by such process was difficult to transfer to hydrolysis. The diazonium salt stained with the unreacted tar like impurities causes uncontrolled increase in temperature. The increase in temperature due to such unreacted tar like impurities also affects safety, control of reaction, control of reaction temperature, yield, and purity of m-cresol final product. Furthermore, the effluent generated in this process cannot be recycled and is majorly discarded.

Therefore, there is a long-standing need to develop a cost effective, economically cheaper, environmentally friendly process and system enabling the process for preparation of m-cresol, and also enabling recycling and reduction of effluent.

### SUMMARY

Before the present system and its components are described, it is to be understood that this disclosure is not limited to the particular system and its arrangement as described, as there can be multiple possible embodiments which are not expressly illustrated in the present disclosure. It is also to be understood that the terminology used in the description is for the purpose of describing the particular versions or embodiments only and is not intended to limit the scope of the present application. This summary is not intended to identify essential features of the claimed subject matter nor is it intended for use in detecting or limiting the scope of the claimed subject matter.

As disclosed herein, the present subject matter relates to a system and method of preparation of m-cresol from m-toluidine in a continuous mode. Further, the present subject matter relates to a system for continuous production of meta-cresol enabling recycle of effluents and reduction in effluent. Further, the present subject matter relates to a process of continuous production of meta cresol.

In one embodiment, a system for continuous production of meta-cresol is disclosed. The system may comprise a gas mixing unit enabled for selectively preparing in-situ N₂O₃ gas. The system may comprise a reactor unit enabled for a meta-toluidine salt preparation. The system may comprise a coiled reactor enabling transfer of meta-toluidine salt within a controlled temperature rage. The system may comprise a diazotization unit comprising a trickle bed reactor unit having a set of packed columns. The system may comprise an excess NOx removal unit. The system may comprise a hydrolysis reactor unit. The system may comprise a layer separation unit connected to a spent acid recovery unit. The system may comprise a meta-cresol collection unit.

In another embodiment, a process of continuous production of meta-cresol is disclosed. The process may comprise a step of obtaining a gas mixture having a maximum amount of in-situ N₂O₃ gas by mixing NO and O₂ gas at a predetermined temperature and flow rate in a gas mixing unit. The process may comprise a step of preparing a meta-toluidine sulfate salt by reacting a predefined amount of meta-toluidine with a predefined amount of sulfuric acid and water in a reactor unit. The process may comprise a step of passing the gas mixture and the meta-toluidine sulfate salt through a diazotization unit comprising a trickle bed reactor unit having a set of packed columns. The process may comprise a step of obtaining a diazonium salt of meta-toluidine sulfate by reacting the meta-toluidine sulfate salt with N₂O₃ in the diazotization unit comprising the trickle bed reactor. The process may comprise a step of removing excess NOx gases by simultaneous addition of sulfamic acid, water, and a diazonium salt of meta-toluidine sulfate to an excess NOx removal unit and thereby suppressing a formation of N₂O₄. The process may further comprise a step of hydrolysing the diazonium salt of meta-toluidine in presence of a premix of water, and toluene in a hydrolysis reactor unit to obtain a crude meta-cresol solution. The process may comprise a step of transferring the crude meta-cresol solution to a layer separation unit. The process may comprise a step of recovering spent acid in a spent acid recovery unit from an aqueous layer separated in the layer separation unit. The process may comprise a step of obtaining a purified meta-cresol in an organic layer from the layer separation unit.

### BRIEF DESCRIPTION OF FIGURES

The detailed description is described with reference to the accompanying Figures. In the Figures, the left-most digit(s) of a reference number identifies the Figure in which the reference number first appears. The same numbers are used throughout the drawings to refer like features and components.

FIG. 1 depicts a system (100) for continuous production of m-cresol, in accordance with an embodiment of the present subject matter.

FIG. 2 depicts a method (200) of preparing a m-Cresol from m-Toluidine, in accordance with an embodiment of the present subject matter.

FIG. 3 depicts a gas mixing unit (101) configured for selective in-situ preparation of N₂O₃ gas, in accordance with an embodiment of the present subject matter.

FIG. 4 depicts a reactor unit (102) enabled for a meta-toluidine salt preparation, and a coiled reactor (103), in accordance with an embodiment of the present subject matter.

FIG. 5 depicts a diazotization unit (104) comprising a trickle bed reactor unit having set of packed columns, in accordance with an embodiment of the present subject matter.

FIG. 6 depicts an excess NOx removal unit (105), in accordance with an embodiment of the present subject matter.

FIG. 7 depicts a hydrolysis reactor unit (106), in accordance with an embodiment of the present subject matter.

FIG. 8 depicts a layer separation unit (107), a spent acid recovery unit (108), and a m-cresol collection unit (109), in accordance with an embodiment of the present subject matter.

### DETAILED DESCRIPTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures or characteristics may be combined in any suitable manner in one or more embodiments.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

It must also be noted that, the singular forms “a,” “an,” and “the” include plural references unless the context clearly dictates otherwise. Although any methods similar or equivalent to those described herein can be used in the practice or testing of embodiments of the present disclosure, the exemplary methods are described. The disclosed embodiments are merely exemplary of the disclosure, which may be embodied in various forms.

Various modifications to the embodiment may be readily apparent to those skilled in the art and the generic principles herein may be applied to other embodiments. However, one of ordinary skill in the art may readily recognize that the present disclosure is not intended to be limited to the embodiments illustrated but is to be accorded the widest scope consistent with the principles and features described herein.

The embodiments, examples and alternatives of the preceding paragraphs or the description and drawings, including any of their various aspects or respective individual features, may be taken independently or in any combination. Features described in connection with one embodiment are applicable to all embodiments, unless such features are incompatible.

In one embodiment a system and process for continuous preparation of m-cresol from m-toluidine is implemented to overcome the major drawback of the conventional m-cresol processes of removal of unreacted sodium nitrite from a diazotization chamber. The inability to remove unreacted sodium nitrite leads to formation of a tar like impurity matter which is difficult to process further or to treat as an effluent at a large-scale production of meta-cresol. Further, the reaction between solid-liquid components causes the formation of by-products and impurities at the diazotization stage. Therefore, a system and process to obtain a pure, clean, economically efficient, environmentally effective, safe and high yield m-cresol is disclosed herewith.

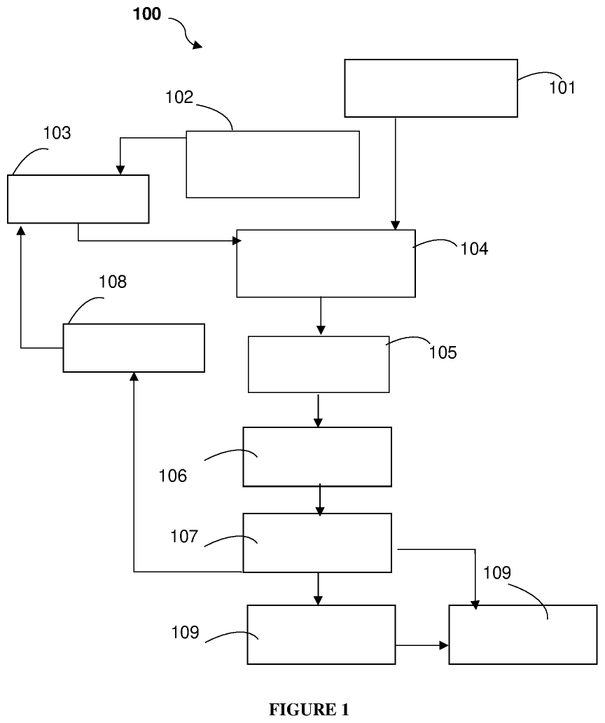

In one embodiment of the present invention, referring to FIG. 1, a system (100) for continuous preparation of a 3-methylphenol (hereinafter may be referred to as ‘m-Cresol’ or ‘meta-cresol’) from m-Toluidine is illustrated in accordance with the embodiments of the present invention. The system may comprise multiple reactors configured to carry out reaction processes such as salt formation, diazotization, hydrolysis, and separation.

In another embodiment, the present disclosure relates to a system and method of preparation of m-cresol and enabling a multiple times recycled use of spent acid i.e., sulfuric acid and other effluent chemicals such as toluene.

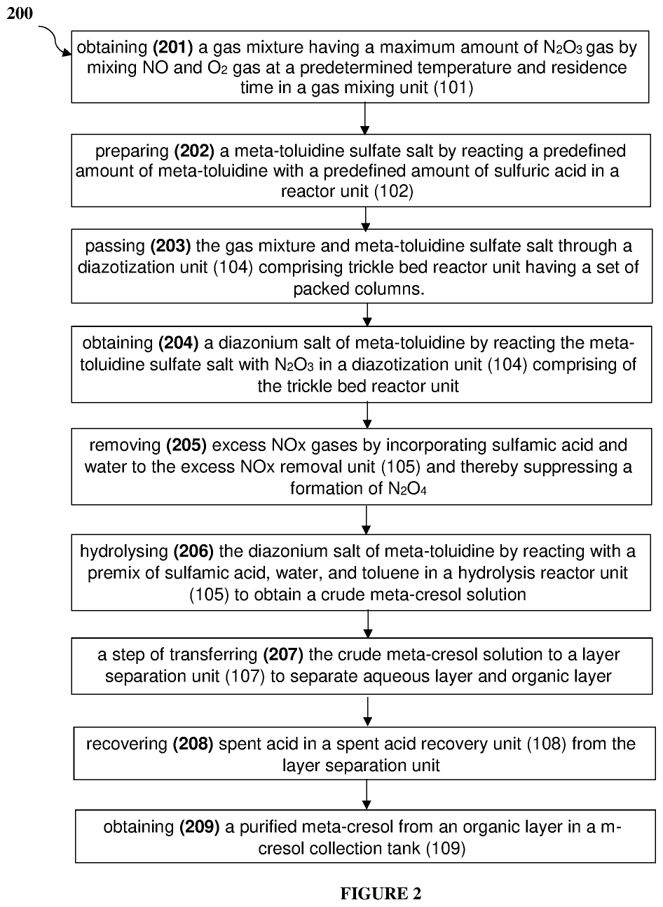

In another embodiment of present invention, referring to FIG. 2, the process (200) of preparation of a m-Cresol from m-Toluidine and NOx reaction may comprise various stages and further divided into plurality of steps. More specifically, the process of preparation of m-cresol may comprise one or more stages such as a stage of preparation of meta-toluidine sulfate salt, a stage of in-situ selective formation of N₂O₃ from NOx mixture, a stage of diazotization and hydrolysis to obtain crude form of m-cresol. The process may be followed by carrying out layer separation in order to separate effluent comprising sulfuric acid and other impurities and further distillation process to get the purified m-cresol. The overall yield of the m-cresol obtained may be between 75-85%, and the reduction of the effluent quantity may be up to 28%-80%.

In one embodiment of the present invention, the first stage may include a preparation of a meta-toluidine sulfate salt by reacting meta-toluidine with sulfuric acid in a reactor unit. In the second stage, the process may include selective preparation of N₂O₃ in a gaseous or liquid form by reacting NO with O₂ gas in a gas mixing unit. In the third stage, a diazonium salt of meta-toluidine is obtained by reacting the meta-toluidine sulfate salt with N₂O₃ in a diazotization unit comprising a trickle bed reactor. In the fourth stage, excess NOx from the diazonium salt of meta-toluidine is removed in an excess NOx removal unit thereby suppressing formation of unwanted N₂O₄. In the fifth stage, hydrolysis of the diazonium salt in presence of water and toluene is performed to obtain meta-cresol and sulfuric acid as an effluent or by-product. In the sixth stage, the process comprises of separating the effluent layer and the m-cresol organic layer in a separation chamber, and by way of distillation of the organic layer obtain a final production of m-cresol having a yield of between 75-85%, and a reduction in effluent quantity by 28%-80%.

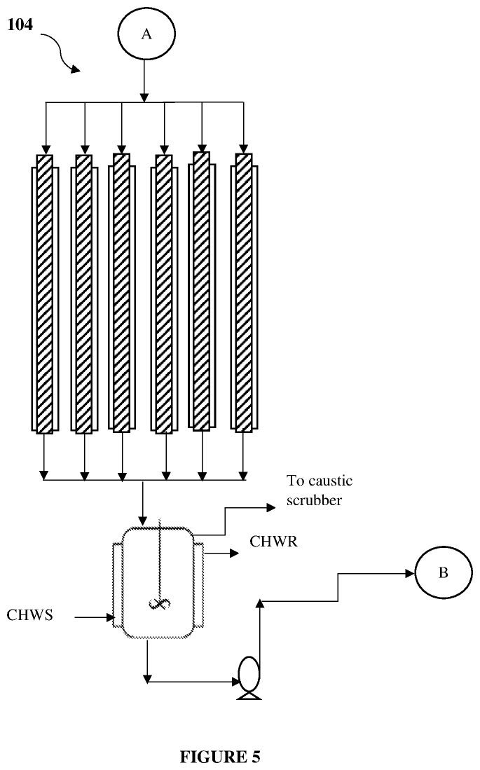

Now referring to FIG. 1, a system (100) for continuous production of meta-cresol enabling effluent reduction is disclosed herewith. Referring to FIGS. 1 and 5, The system (100) may comprise a gas mixing unit (101) enabled for selectively preparing in-situ N₂O₃ gas.

In one embodiment, the gas mixing unit (101) may involve a plurality of parallel/series elevation reactions taking place with individual kinetics-HOR and effect on next followed reaction performance. Each reaction control parameters are different and opposite to another. The said series of elevation reactions is more complex, the below set of reactions show the reaction of NO and O₂ in a gas mixing unit (101) till the formation of NOx gases and its effect on diazotization salt formation.

2NO+0.5O₂→NO₂

NO+NO₂→N₂O₃ or NO2+NO₂→N₂O₄

N₂O₃+H₂O→2HNO₂ or N₂O₄+H₂O→HNO₂+HNO₃

MT salt+HNO₂→Diazo salt or MT salt+HNO₂+HNO₃→Diazo salt+impurity

MT+Diazo salt→Impurity

In one embodiment, N₂O₃ or N₂O₄ formation depends upon whether NO₂ react with NO or another NO₂ molecule in gas phase. A higher mole concentration of NO in gas phase is observed to be increasing formation chances of N₂O₃ (i.e., NO:O₂ mole).

N₂O₃ and N₂O₄ formation rate increase with temperature decrease. However, with decrease in temperature, relative N₂O₄ formation rate is always more than N₂O₃ formation rate (because more NO₂ formation and better chance to react NO₂ with NO₂).

In one embodiment, for diazotization, a maximum mole of HNO₂ is required. A reaction with water and N₂O₃ gives 2 moles of HNO₂, and N₂O₄ giving one mole of HNO₂, and one mole of HNO₃ which is not desirable as being not useful for the formation of diazo product. Therefore, it is observed that to produce more HNO₂, formation of N₂O₃ is desirable.

In one embodiment, to increase N₂O₃ formation by virtue of kinetics i.e., to consume it faster than a rate at which the N₂O₃ may be produced, as a kinetic equilibrium continuously shifts from N₂O₄ to N₂O₃. By reducing the formation of N₂O₄ and improvising production of N₂O₃, at an actual 75-85% product yield of meta-cresol may be obtained.

It is observed that high temperature mixing caused low reaction rate of NO and O₂ where temperature range is not controlled below 35 degrees. Further, mixing of NO and O₂ at lower temperature incurred more residence time. Therefore, an optimization of reaction temperature and reaction residence time was carried out to achieve low temperature mixing between 5 degrees to 25 degrees having high reaction rate of NO and O₂.

Further, the resident time of mixing NO and O₂ is maintained within the range of 0.1 minutes to 15 minutes and preferably 0.5-10 minutes in the gas mixing unit (101). When NOx comprising NO and O₂ are mixed with optimized reaction conditions, a faster consumption of N₂O₃ shifting equilibrium to generate maximum quantity of N₂O₃ in a gas mixing unit (101) was achieved.

Further, removal of heat of reaction from NO and O₂ reaction may need some heat transfer area A1 having a total volume V1. This V1 should be adequate to complete the reaction at a predetermined temperature of 5 to 25 deg. In other words, area should be adequate to remove heat, volume should be adequate for residence time from same heat transfer area should be adequate to complete reaction.

In still another embodiment, a reaction complexity in a production process of m-cresol is described. It is observed that reaction rate of NO with O₂ increase with decrease in temperature and produce N₂O₃:N₂O₄ in particular ratio. A much stronger tendency for NO₂ to dimerize and for N₂O₄ rather than formation is 20.9 times higher than N₂O₃. The heat of reaction including HNO₂ from N₂O₃ was observed at −1254 KJ/mole including HNO₂ from N₂O₃. However, therefore selective in-situ formation of N2O3 over N2O4 is achieved by configuring the reactant flow rate, temperature, and resident time conditions.

In one embodiment, the gas mixing unit (101) comprises a compressed NO gas inlet and a O₂ gas inlet from oxygen gas storage tank. The inlets of both the NO and O₂ gases are connected to respective heat exchanger units to optimize the reaction temperature to 5-25 deg and further optimize the flow rate of mixing.

The gas mixing unit (101) is enabled to mix O₂ and NO for selectively preparing N₂O₃ at a predetermined flow rate. In one embodiment, a particular ratio of NO and oxygen was maintained as 7:1 to 4:1 and more preferably 6:1 at temperature 5-25° C., and reaction time of 0.1 minutes to 10 minutes.

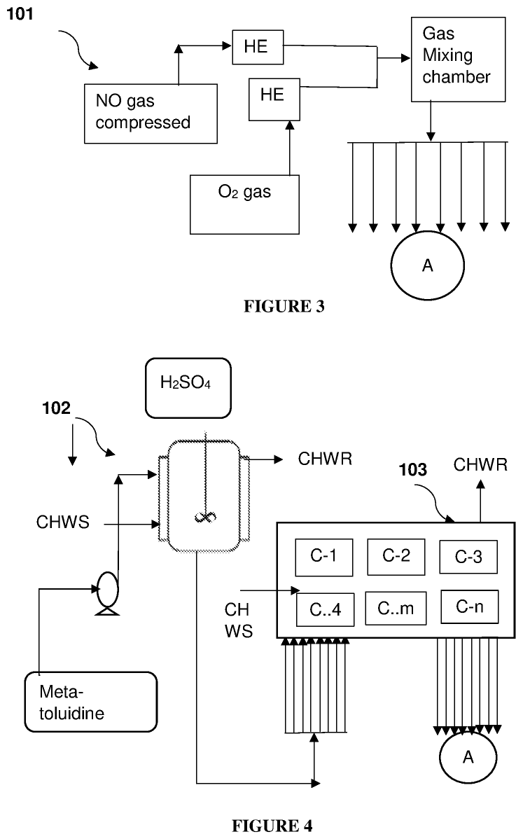

Referring to FIGS. 1 and 3, the NO and oxygen gases may be mixed in the gas mixing chamber of the gas mixing unit (101) to selectively form N₂O₃ as depicted in FIG. 3. Further, the in-situ N₂O₃ formed may be enabled to be transferred to the diazotization unit (104) (process flow depicted as ‘A’ in FIGS. 4 and 5) via a plurality of conduit units. In one embodiment, the number of conduit units may be dependent upon the number of packed columns of the diazotization unit (104).

In another embodiment, referring to FIG. 1 and FIG. 4, the system (100) may comprise a reactor unit (102) enabled for a meta-toluidine salt preparation. The system (100) may comprise a coiled reactor (103) connected to the reactor unit (102) and to pass the meta-toluidine sulfate salt to the diazotization unit (104). The coiled reactor (103) may comprise a set of cooling coils C1, C2, C4, . . . C_{N} for pre-cooling the meta-toluidine (MT) salt solution. In an embodiment, the pre-cooling temperature of the set of cooling coils may be maintained as 5-20° C. and preferably between 10-15° C. In one embodiment, the number of cooling coils of the coiled reactor (103) may be dependent upon the number of packed columns of the diazotization unit (104) to which the coiled reactor (103) is connected.

In one embodiment, referring to FIG. 1 and FIG. 5, the diazotization unit (104) comprises of a first section containing a trickle bed reactor unit having a set of packed columns, and a section containing a diazotization salt collection chamber. In an optional embodiment, the trickle bed reactor and the diazotization salt collection chamber collectively may be formed as a single member diazotization unit.

In one embodiment, the set of packed columns in the trickle bed reactor may be enabled to transfer the meta-toluidine salt, and a mixture of NO and O₂ gas towards the diazotization chamber.

In one embodiment, the set of packed columns in the trickle bed reactor may be a high efficiency packing columns enabling increase in weight and mass transfer efficiency of the overall production of meta-cresol.

In one embodiment, the number of packed columns may vary between 2-9, preferably 4-7, and may further vary as per the reaction scale up. It should be noted herein that the increase in number of packed column reactors as per the reaction scale does not affect the efficiency of the diazotization unit and % yield of the final meta-cresol product. The set of packed columns may have different types of packings selected from, but not limited to, structured and random packings. Further, the material of construction of the packings may be glass, ceramic, and steel, etc. Furthermore, the random packings may be any of, but not limited to, glass beads, pall ring, and Raschig rings, and the like.

In an exemplary embodiment, a diameter of packed column is between but not limited to 2-5 cm and more preferably between 3 to 4 cm with structured packings. In another embodiment, tube diameter is between 2.4 to 3.4 cm and column height between 90-120 cm. Therefore, a ratio of column diameter:column height may be adjusted as 1:60 to 1:20 and preferably 1:45 to 1:24. In one embodiment, for abovementioned exemplary specifications of packed column, a surface area achieved may be up to 700-2500-meter Sq surface area for per m3 of volume. In one embodiment, the set-up of excess NOx removal unit (105) may be connected with a set of packed columns, wherein a packing height vs packing diameter limitation as disclosed above with height efficiency packing increases wetting and mass transfer efficiency of the diazotization unit (104) to the excess NOx removal unit (105).

In one embodiment, a reaction between in-situ N₂O₃ and m-toluidine salt may take place in the packed columns in the trickle bed reactor of the diazotization unit (104) enabling maximum amount of in-situ formation HNO₂ and thereby formation of diazotized mass in the diazotization salt collection chamber of the diazotization unit (104). The trickling mechanism of the diazotization unit reduced formation of impurity, and improved the overall yield in a continuous mode, and further achieved slow, steady, and consistent reaction between N₂O₃ (enabling formation on-line formation of HNO₂) with m-toluidine sulfate salt. It should be noted that, the variation in the number of packed columns in the trickle bed reactor of the diazotization unit (104) as per reaction scale requirements improves safety by implementing the continuous reaction in a controlled process at a controlled temperature.

As shown, the gas mixing unit (101) and the reactor unit (102) are coupled with the diazotization unit (104) comprising of trickle bed reactor unit (depicted by ‘A’).

Furthermore, the diazotization unit (104) is connected to an excess NOx removal unit (105). The excess NOx removal unit (105) comprises an inlet for sulfamic acid, and the diazotization mass formed in the diazotization unit (104). In one embodiment, the sulfamic acid and the diazo salt has a parallel input to the excess NOx removal unit (105). In one embodiment, the excess NOx removal unit (105) may be a microreactor unit enabled for removal of excess NOx and suppressing formation of N₂O₄ gas responsible for m-cresol yield reduction. In another embodiment, the excess NOx removal unit (105) is a continuous plug flow reactor enabled for static mixing and removal of excess NOx and suppressing formation of N₂O₄ gas by static mixing mechanism.

Furthermore, the excess NOx removal unit (105) is configured for removing of excess NOx by reacting the diazonium salt of meta-toluidine with a sulfamic acid solution. The excess NOx removal unit (105) is incorporated before a hydrolysis reactor unit (106) to suppress formation of N₂O₄ which degrades the quality of diazotization salt of m-toluidine sulfate. The excess NOx removal unit (105) also enables the reduction in formation of any undesired side-products and selectively improves m-cresol yield in the hydrolysis reactor unit (106).

In one embodiment, referring to FIG. 1 and FIG. 7 the system (100) may comprise the hydrolysis reactor unit (106) enabling a crude production of meta-cresol from the diazotized meta-toluidine salt solution. In one embodiment, the hydrolysis reactor unit (106) may be configured to hydrolyse crude diazotization mass to convert diazo meta-toluidine salt to crude meta cresol. The hydrolysis reactor unit (106) also enables increment in conversion of diazo meta-toluidine salt to meta cresol.

In one embodiment, the hydrolysis reactor unit (106) is a continuous stirred tank type reactor enabling maximum conversion of the diazotized meta-toluidine salt to meta-cresol in presence of toluene. In one embodiment, the system (100) may comprise a toluene feeding and recovery unit wherein the excess toluene from the hydrolysis may be recovered and fed back to the hydrolysis reactor unit (106). In one embodiment, the toluene recovery unit and toluene feeding unit may be a different tank or a common tank enabling recycle of toluene. In one embodiment, about 75-85% and preferably 75-80% yield of m-cresol is obtained by continuous hydrolysis in a continuous stirred tank reactor (CSTR) with effluent recycle at least 6 times. In one embodiment, the recycle of an effluent reduced the spent acid quantity by 28-80%. In one embodiment, spent acid may be referred to a mixture of sulfuric acid, nitric acid, water, and the related organic compounds, and sulfuric acid.

In one embodiment, the system (100) may comprise a layer separation unit (107), wherein the layer separation unit (107) is enabled to separate a pure meta-cresol from the crude meta-cresol in an organic layer. The layer separation unit (107) may be configured for separating an effluent layer and m-cresol (organic) layer, and further obtaining a pure m-cresol product by way of distillation of m-cresol (organic) layer in a distillation unit (110), wherein the yield of a final production of m-cresol is between 75-85%, and wherein the reduction in effluent quantity up to 28%-80% is observed. Also, the layer separation unit may be attached to a spent acid collection unit (108) which may be further connected to the reactor unit (102), the hydrolysis reactor unit (106) and the diazotization unit (104) to recycle the spent acid, i.e., sulfuric acid. In one embodiment, a spent acid from the aqueous layer of the layer separation unit is recovered and separated to be collected in a spent acid collection tank (108).

In one embodiment, layer separation unit (107) of the system (100) may be further connected to a meta-cresol collection unit (109). In one embodiment, an organic layer comprising m-cresol is transferred to distillation unit (110) enabled for collection of pure form of meta-cresol having a purity of 99.9%.

In one embodiment, a continuous production process of m-cresol in a modified continuous m-cresol preparation apparatus is disclosed herein. The process of continuous production of m-cresol involves a NOx route and achieves a m-cresol yield about 75-85% and recycle of aqueous layer of spent acid i.e., sulfuric acid for 2-10 times and preferably 2-6 times.

In one embodiment, the system (100) for the continuous large-scale production of m-cresol is configured in such way that the production scale can be amplified in an expansive manner by changing number of conduit units. One of the advantages of the system (100) is that the gas mixing unit (101), and the reactor unit (102) are affixed to the diazotization unit (104) (comprising trickle bed reactor having packed columns) via one or more of plurality of conduit connections which results in improving reaction safety, enabling higher temperature reactions, consistent spent acid recovery and consistent m-cresol yield. Also, in the same continuous system (100), two or more conduit connections may be implemented to achieve any production size without hampering the overall reaction yield between 75-85% and quality.

Now referring to FIG. 2, a process (200) of continuous production o meta-cresol is disclosed. The process (200) comprises one or more steps implemented with the system (100). In one embodiment, the said process (200) may be an end-to-end multistage continuous process.

In a first stage, an in-process preparation of nitrous acid by reacting NOx gases such as NO (g) and NO₂ (g) with water may be performed. The process (200) may comprise a step of obtaining (201) a gas mixture having a maximum amount of N₂O₃ by mixing NO and O₂ gas at a predetermined temperature and predetermined flow rate in the gas mixing unit (101). In one embodiment, the predetermined temperature in the step of obtaining (201) is maintained as 15-16° C., and preferably 14-20° C. In one embodiment, a molar ratio of O₂:NO may be maintained between 1:10 to 1:4 and preferably 1:6.

In a second stage, a preparation of meta-toluidine sulfate salt may be carried out by reacting meta-toluidine with sulfuric acid. The process (200) may comprise step of preparing (202) a meta-toluidine sulfate salt by reacting a predefined amount of meta-toluidine with a predefined amount of sulfuric acid, and water in the reactor unit (102). In one embodiment, the reaction scheme of preparation of meta-toluidine sulfate salt by reacting meta-toluidine with sulfuric acid is depicted as below.

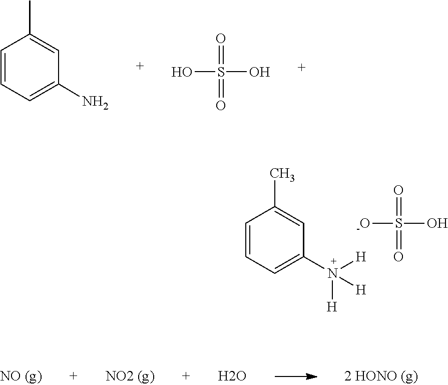

In one embodiment, the reaction of meta-toluidine with a predefined amount of sulfuric acid in presence of water in the reactor unit (102) may be carried out at 15-25° C. and preferably 15-20° C. by exothermic reaction mechanism. In one embodiment, the strength of sulfuric acid may be 30%-98% sulfuric acid and preferably 98%. In one embodiment, a molar ratio of Meta-toluidine vs 98% sulfuric acid may be maintained as 1:6 to 1:3 and preferably as 1:4.78. Furthermore, a water to m-toluidine salt ratio may be maintained as 1:59.

In a third stage, the said gas mixture comprising selective in-situ N₂O₃ is reacted with meta-toluidine sulfate salt to carry out diazotization and to obtain meta-toluidine diazonium salt in the diazotization unit (104). In one embodiment, temperature in the diazotization unit (104) may be maintained between 5-25° C. and specifically 15-20° C. In this stage, the process (200) may comprise a further step of passing (203) the gas mixture and meta-toluidine sulfate salt through the diazotization unit (104) comprising a trickle bed reactor unit having a set of packed columns.

The process (200) is optimized by involving a plurality of trials with different packed column tubes comprising various type of packings in order to increase a m-cresol yield capacity as compared to the conventional systems/methods.

In an embodiment, the overall effluent quantity reduction at source is achieved up to 28%-80% and specifically up to 28%-50%. Further, the incorporation of packed columns improved recycle of acid by avoiding formation of the black viscous tar impurity of meta-toluidine and obtain maximum conversion at diazotization stage and to achieve a controlled and safe reaction process.

The process (200) may comprise further step of obtaining (204) a diazonium salt of meta-toluidine by reacting the meta-toluidine sulfate salt with N₂O₃ in the trickle bed reactor unit of the diazotization unit (104).

The process (200) may further comprise a step of removing (205) excess NOx gases by simultaneous transfer of diazotized met-toluidine salt, water and sulfamic acid to an excess NOx removal unit (105) and thereby suppressing formation of N₂O₄ and further enabling in-situ formation of HNO₂ at a point of diazotization reaction to obtain a higher yield of diazotization product. In one embodiment, temperature of the excess NOx removal unit (105) may be maintained between 0-5° C.

In one embodiment, the reaction scheme of preparation of diazotized salt of meta-toluidine by reacting meta-toluidine sulfate salt with NOx is depicted as below.

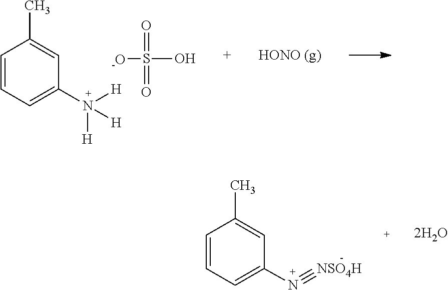

In a fourth stage, a continuous hydrolysis of the meta-toluidine diazonium salt is carried out in presence of water in the hydrolysis unit (106), by adding toluene as a solvent to obtain a meta-cresol crude product. The process (200) may comprise a step of hydrolysing (206) the diazonium salt of meta-toluidine at a predetermined temperature range of 80-85° C. by reacting with a premix of water, and toluene in a hydrolysis reactor unit (106) to obtain crude meta-cresol solution. The process as disclosed in FIG. 2 enabled reduction in effluent quantity by 28%-80%. In the hydrolysis step further to diazotization, 75%-85% yield of m-cresol is obtained in a continuous process.

The process (200) may comprise a step of transferring (207) the crude meta-cresol solution to a layer separation unit (107) to separate aqueous layer and organic layer by toluene washing and Na₂CO₃ neutralization at the temperature range of 20-25° C.

In one embodiment, the process (200) may comprise a step of recovering (208) spent acid in a spent acid recovery unit (108) from the layer separation unit. In one implementation of the process (200), the said spent acid in effluent an aqueous layer may be recovered and recycled at least for 6+1 times to achieve an economically viable, safer, and environment friendly process. Further, in one embodiment the spent acid effluent may be treated with activated charcoal or carbon to complete colour removal and to obtain an impurity free spent acid for recycle and reuse in the process.

The process (200) may comprise a step of obtaining (209) a purified meta-cresol from an organic layer in a m-cresol collection tank (109) from the layer separation unit (107).

In yet another embodiment, 28-80% and preferably 28-50% of effluent quantity reduction at source has been achieved with at least 6 times of effluent recyclability with 75-85% and probably up to 79% yield of m-cresol.

In another embodiment, a process of treating effluent formed in the process of production of m-cresol may comprise of treating the effluent by activated carbon, wherein the effluent may comprise diazotization by-products such as sulfuric acid; and obtaining a clear solution of the effluent configured for recycling and re-use.

The present invention is further described by the following examples as proof of concept:

### EXPERIMENTAL DETAILS

In one implementation of the present invention, several factors were studied, which are associated to stoichiometric balance of the reactants, molar ratios of the one or more components, optimization of concentration, recovery, yield, recycle mechanism, contact time, reaction temperature, distillation techniques, reaction calorimetry, and product work-up technique optimization.

### Example 1 (Selective Formation of N₂O₃ in the Process of Preparation of m-Cresol)

The Example 1 of the present disclosure referred to a process step of gas mixing of NO and O₂ gas to selectively obtain N₂O₃ by maintaining the operating conditions and flow rate of mixing in a process of preparation of m-cresol. In one embodiment, a molar ratio of Oxygen:NO is maintained as 1:10 to 1:4 and preferably 0.27:1.62 to 0.3:2.

The process may involve in-situ selective formation of N₂O₃ from NOx mixture, a stage of diazotization and hydrolysis to obtain crude form of m-cresol. The resident time of mixing NO and O₂ is maintained within the range of 0.1 minutes to 15 minutes and preferably 0.5-10 minutes in the gas mixing unit (101). Further, removal of heat of reaction from NO and O₂ reaction may need some heat transfer area A1 having a total volume V1. This V1 should be adequate to complete the reaction at a predetermined temperature of 5 to 25 deg and preferably 13-16° C. In one embodiment, a ratio of feed flow rate of NO with oxygen is 0.6-1: 4-6. In another embodiment, a flow rate of NO was maintained between 200-400 gm/hr (291.5) gm/hr. min 200 gm/hr, optimum 288 gm/hr when transferred to a trickle bed reactor comprising a set of 6 packed column reactors.

Further a m-toludine sulfate salt is prepared by reacting m-toluidine with 98% sulfuric acid in presence of water. The flow rate of m-toluidine salt is maintained about 16-27 ml/min (for a single coil tube) when added to a diazotization unit from a coiled reactor for the formation of diazotization salt of m-toluidine sulfate.

The process of preparation of m-cresol obtained by hydrolysis of diazotization salt of m-toluidine sulfate is a scale up process carried out in a continuous mode. The yield of the m-cresol obtained by hydrolysis of diazotization salt of m-toluidine sulfate may be observed between 75-85%, and wherein the reduction of the effluent quantity may be achieved up to 28%-80%.

### Example 2 (Optimization of Sulfuric Acid as Starting Material)

In an embodiment, the m-cresol production process has been developed to work in a continuous mode. It has been observed that the continuous mode process is economic and environmentally complacent.

In one embodiment, a reaction optimization in the third stage of diazotization is illustrated. In this example, a change in molar equivalents of sulfuric acid in the diazotization reaction on a post hydrolysis yield of m-cresol is studied. The toluene solvent may be used as a solvent in hydrolysis stage. An effect change of molar equivalents of sulfuric acid between a range of 2-5 mole eq. was studied. Specifically, a molar ratio of Meta-toluidine vs 98% sulfuric acid was optimized be maintained as 1:6 and preferably 1:4.78.

### Stoichiometric and Balanced Chemical Equation:

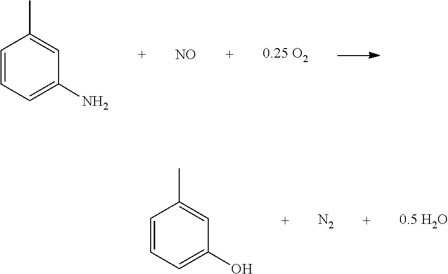

### Stoichiometric and Balanced Chemical Equation:

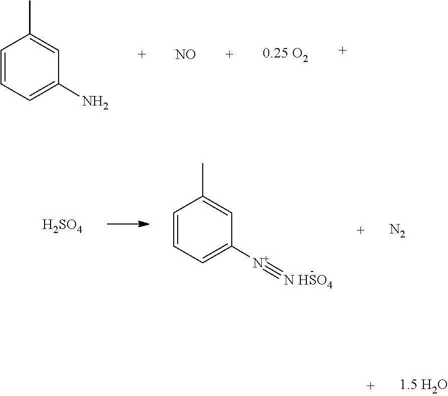

TABLE 1

| Experiment no. | 1 | 2 | 3 |
|---|---|---|---|
| total feed size (g), | 107 g | 107 g | 107 g |
| Meta-toluidine | | | |
| Sulfuric acid | 645.00 g; | 1129.0 g; | 1059.0 g; |
| Qty.g (Equivalent.) | (2.0 moles) | (3.5 moles) | (4.77 moles) |
| Temperature (° C.) | 15-20 | 15-20 | 15-20 |
| Unreacted MT | 3.07 | 0.98 | 0.53 |
| % w/w in Diazo | | | |
| intermediate product | | | |
| m-cresol Yield | Not Hydrolyzed | Not Hydrolyzed | 681 g/80.69% |

Referring to Table 1, it was observed that the more unreacted starting material was obtained when the less mole equivalents of sulfuric acid was used resulting in the production of impure meta-toluidine diazotization salt. The unreacted meta-toluidine diazotization salt concentration is higher in less mole eq. of sulfuric acid solution. Increase in moles of sulfuric acid in the diazotization stage, led to decrease in tar like impurity. Therefore, the molar ratio of sulfuric acid in the diazotization step was optimized to 4.77 moles against 1 mole of MT in order to obtain the improved yield of a diazotization product in presence of toluene as a hydrolysis solvent.

In the third stage of diazotization, the meta-toluidine is reacted with NOx in presence of sulfuric acid in a diazotization unit comprising a tricked bed reactor having a set of packed column reactors and followed by hydrolysis to obtain m-cresol yield up to 80%.

### Example 3 (Optimization of Oxygen Quantity)

In one embodiment, a reaction optimization in the third stage of diazotization by minimizing the impurity and improving the meta-cresol yield by specifically optimizing the mole eq. of oxygen is illustrated in Table 2. In this trial, the temperature parameter was kept constant between 15-20 degree Celsius.

TABLE 2

| Batch no. | 4 | 5 | 6 |
|---|---|---|---|
| Batch size (g), | 535 g | 428 g | 428 g |
| Meta-toluidine | | | |
| Oxygen (100%) g | 43.2 g; (0.27) | 37.12 g; (0.29) | 42.24 g; (0.33) |
| (mole Equivalent) | | | |
| Temperature | 15-20 | 15-20 | 15-20 |
| (° C.) | | | |
| m-cresol Yield | 78.35% | 89.87% | 78.69% |

Referring to Table 2, it was observed that increase in molar equivalents of oxygen in diazo process led to increase in impurity having a tar like form. Further, unreacted meta-toluidine was observed when less mole equivalents of oxygen was utilized at diazotization stage. The highest m-cresol yield with minimum impurity has been obtained when the molar equivalents of oxygen in the gas mixing chamber was adjusted between 0.25-0.29 mole equivalents i.e., up to 79%.

### Meta-Toluidine Salt Preparation

Referring to FIG. 1, a predefined amount of water was added to a reactor unit, and cooling the mass for 10° C. The sulfuric acid was charged in the reactor unit and the temperature was maintained at 25° C. The reaction mass was checked and if observed unclear, the reaction mass was made clear by heating the reaction mass to 40° C. Further, the reaction mass heated was cooled up to 25° C. In another embodiment, the temperature the reactor unit was maintained between 12-15° C.

### Packed Bed Set Up Arrangement.

In an embodiment, referring to FIG. 1 and FIG. 5, a trickled bed reactor comprising a jacketed packed glass columns having dimension of 1200 mm height and 34 mm internal diameter is illustrated. A specific structural packing till height of 1200 mm was loaded to each of the six packed columns. A chiller unit was attached with the columns and a cooling set point was applied at −12° C.

In one embodiment, a most unreacted meta-toluidine was observed when reaction was performed at <15° C.

### Continuous Diazotization Process of Meta-Toluidine Salt

In one embodiment, referring to FIG. 5, a set of six packed columns was attached in the trickled bed reactor wherein each packed column was enabled for monitoring and adjustment of temperature parameter. In one embodiment, the diazotization system was pre-cooled by applying a chiller system. Moreover, the temperature of the diazotization unit after packed columns was maintained between 15-18° C. In one embodiment, the diazotized meta-toluidine was obtained at about 6700-6800 gm for a 1 liter reaction.

It was also observed that other solid-liquid by-products were not generated. The nitrogen generated as a byproduct was recovered through an escape vent. Water was used as cleaning solvent in this reaction. Further, the work-up conditions were followed by using an organic solvent layer washing. Therefore, the reaction temperature was optimized to be maintained between the range of 15-20° C.

### Example 3 Excess NOx Removal and Suppression of Formation of N₂O₄ in a Diazo Mass

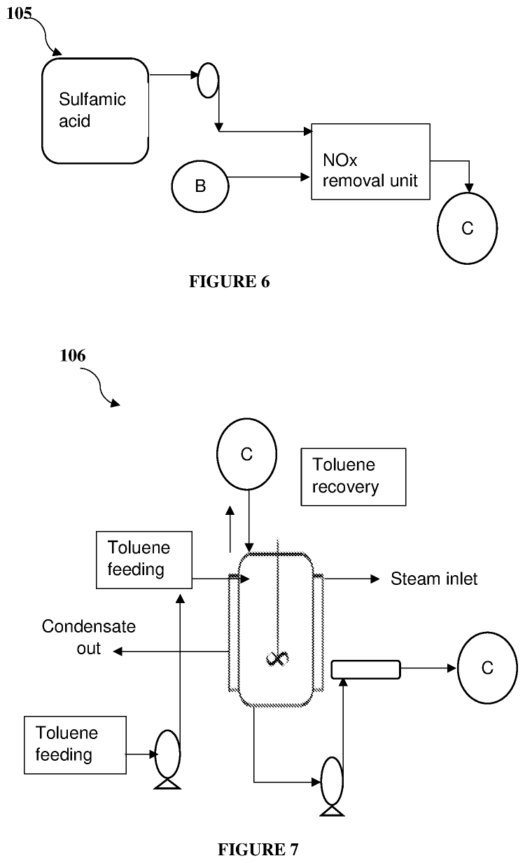

In an embodiment, referring to FIG. 1 and FIG. 6, in the next step, the diazotized meta-toluidine salt was pumped to a microreactor unit. The diazotization reaction took place in the diazotization unit and a meta-toluidine salt diazotized product was collected in the diazotization mass collection unit and then transferred to the excess NOx removal unit (microreactor). The excess NOx removal unit is a plug flow static reactor enabled for parallel addition of sulfamic acid and diazotization mass.

Also, a sulfamic acid solution was fed to the microreactor unit for excess NOx removal present along with the diazotized meta-toluidine salt at predefined flow rate. In one embodiment, both the components were mixed through pump as per above mentioned flow rate through the microreactor unit having coiled arrangement for static mixing and excess NOx removal reaction. In one embodiment, a coiled arrangement was enabled to maintain 0° C. In one embodiment, the coil outlet of the excess NOx removal unit (microreactor unit) removed excess NOx from the diazotized meta-toluidine salt. In one embodiment, the coil outlet was further connected to a continuous hydrolysis unit. The said continuous hydrolysis unit may be a continuous stirred tank reactor.

### Continuous Hydrolysis Unit:

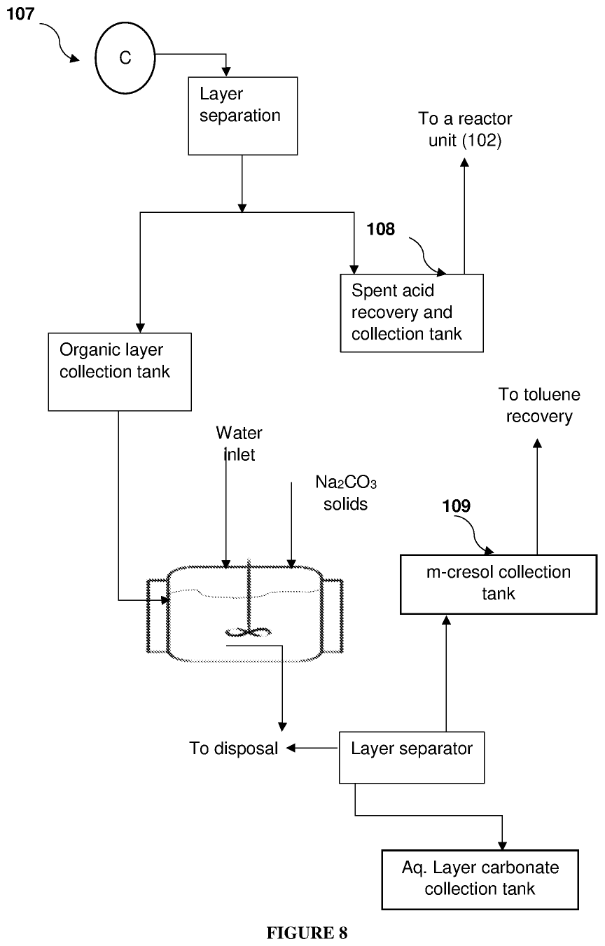

In one embodiment, again referring to FIGS. 1, 4 and 8, a system enabled for process of continuous hydrolysis of diazotized meta-toluidine salt in a continuous stirred tank reactor is disclosed. Further the said the diazotized meta-toluidine salt was passed to a hydrolysis unit. The hydrolysis of the diazotized m-toluidine sulfate salt was carried out in a continuous preparation of m-cresol in a continuous stirred tank reactor. In the said continuous stirred tank reactor, a predefined amount toluene was charged and heated till 80-85° C. In one embodiment, a predefined residence time for hydrolysis was adjusted between 90-110 min and specifically 100 min.

The overflow of a hydrolysis reaction was collected in a m-cresol collection tank. Further, the organic and aqueous layer separation was observed. The aqueous layer was further washed with toluene to further separate out organic layer and sulfuric acid (spent acid) up to six times. Further the said organic layer was washed with sodium carbonate solution till pH of organic layer gets neutral. The aqueous layer comprising spent acid was separated from the reactor. In one embodiment, the meta-cresol was formed and collected in an organic layer. The said organic layer comprising a crude meta-cresol was distilled in presence of Triethanolamine to obtain 75%-85% yield of m-cresol.

### Example 4 (Recovery of Spent Acid from Aqueous Layer)

In an example, a reusability of solvent at least for 7 times (i.e. fresh effluent+6 times recycle) reusable recycle is optimized. The reusability of an aqueous layer of hydrolysis reaction mass of m-cresol for minimum fresh+6 consecutive cycles was observed and represented in Table 3. At 7 times recycle of spent acid to a m-toluidine sulfate salt forming reactor unit does not affect the consistency in final yield of m-cresol.

The Example 4 of the present disclosure referred to the effluent recyclability cycles of meta-toluidine as depicted in Table 3.

TABLE 3
Effluent recyclability trials - Continuous diazotization followed
by continuous hydrolysis

| | Spent acid | | | |
|---|---|---|---|---|
| | input to | | | |
| | meta | | | |
| Set | toluidine | Aq Effluent | Unreacted MT | m-cresol |
| (1-4 trials) | reactor | recycle from | % w/w | % yield |
| S1 | 107 | Fresh | 0 | 80.6 |
| S2 | 107 | S1 | 0 | 80.46 |
| S3 | 107 | S2 | 0 | 78.96 |
| S4 | 107 | S3 | 0 | 79.28 |
| S5 | 107 | S4 | 0.01 | 77.91 |
| S6 | 107 | S5 | | 77.81 |
| S7 | 107 | S6 | 0.2 | 79.02 |
| S8 | 107 | S7 | 0.83 | 72.53 |

Referring to Example 4 and Table 3, a 6+1 times effluent recycle trials in continuous end to end mode where diazotization and hydrolysis are involved, where in the 7^{th} recycle time more of the unreacted meta-toluidine was observed and thereby reduction in m-cresol yield is observed. Therefore, at least 6 times of sulfuric acid and effluent recycle in a continuous reaction is carried out with average 79% yield.

In one embodiment, a recyclability of the aqueous layer to recover spent sulfuric acid was studied for 6 cycles. Further, the yield and quality of meta-cresol obtained till 6 recycles was consistent. Also, the recovered toluene during the distillation process is recycled in next batch and found same throughput. Further, recycling of an aqueous layer done in six cycle and found consistent results with respect to m-cresol yield. The overall yield obtained the 6 times recycled aqueous layer is 78-79%.

In accordance with embodiments of the present disclosure, the m-Cresol preparation method described above may have following advantages including but not limited to:
economic viability, safety, and environment friendly features of the process is optimized by using trickle bed reactor instead of using aqueous solutions in the diazotization step
Simple and easy recovery of reactants
Cost-effective-continuous process for preparation m-cresol wherein MT diazonium salt is prepared using MT sulfate salt solution and NO+O2(g) [gas-liquid phase reaction] using trickle bed reactor, followed by Hydrolysis
Recycling of effluent multiple times for at least 6 times, and thereby reducing of effluent of about 28%-50% as compared with conventional process for preparation of m-cresol such as using SNI and others.
With the increase in recycling of effluent, percentage of reduction of effluent also increases thereby reducing sulfuric acid quantity and the overall cost.

Various modifications to the embodiment will be readily apparent to those skilled in the art and the generic principles herein may be applied to other embodiments. However, one of ordinary skill in the art will readily recognize that the present disclosure is not intended to be limited to the embodiments illustrated but is to be accorded the widest scope consistent with the principles and features described herein.

The foregoing description shall be interpreted as illustrative and not in any limiting sense. A person of ordinary skill in the art would understand that certain modifications could come within the scope of this disclosure.

The embodiments, examples and alternatives of the preceding paragraphs or the description and drawings, including any of their various aspects or respective individual features, may be taken independently or in any combination. Features described in connection with one embodiment are applicable to all embodiments unless such features are incompatible.

1. A system for continuous production of meta-cresol comprising:
a gas mixing unit enabled for selectively preparing in-situ N₂O₃ gas;
a reactor unit enabled for a meta-toluidine salt preparation;
a diazotization unit comprising a trickle bed reactor unit having a set of packed columns, wherein the trickle bed reactor unit is enabled for diazotization of m-toluidine salt in presence of in-situ N₂O₃ gas to obtain diazotization salt of meta toluidine sulfate;
a hydrolysis reactor unit for conversion of the diazonium salt of meta toluidine sulfate to meta-cresol;
a layer separation unit;
a spent acid recovery unit enabled to recover spent acid from an aqueous layer of the layer separation unit, and
a meta-cresol collection unit.

2. The system as claimed in claim 1, further comprising a coiled reactor unit connected to a reactor unit enabled for cooling and maintaining temperature of m-toluidine salt to a predetermined range of 5° C.

3. The system as claimed in claim 1, further comprising an excess NOx removal unit enabled for removal of excess NOx from the diazotization salt of meta toluidine sulfate.

4. The system as claimed in claim 1, wherein the hydrolysis reactor unit is a continuous stirred tank type reactor.

5. The system as claimed in claim 1, wherein the set of packed columns comprising different types of packings selected from structured and random packings.

6. The system as claimed in claim 1, wherein set of packed columns is having a column diameter to column height ratio in a range of 1:60 to 1:20.

7. The system as claimed in claim 1, wherein the gas mixing unit is enabled to mix O₂ and NO for selectively preparing in-situ N₂O₃.

8. The system as claimed in claim 3, wherein a ratio of flow rate of NO with oxygen is 0.6-1:4-6.

9. The system as claimed in claim 1, wherein the spent recovery unit is configured to recover and recycle spent acid at least for six times.

10. The system as claimed in claim 1, further comprising a distillation unit for distillation of meta-cresol in an organic layer and to obtain a purified meta-cresol.

11. A process of continuous production of meta-cresol, comprising:
obtaining a gas mixture having a maximum amount of N₂O₃ gas by mixing a predetermined ratio of NO and O₂ gas at a predetermined temperature and residence time in a gas mixing unit;
preparing a meta-toluidine sulfate salt by reacting a predefined amount of meta-toluidine with a predefined amount of sulfuric acid in a reactor unit;
passing the gas mixture and the meta-toluidine sulfate salt through a diazotization unit comprising a trickle bed reactor having a set of packed columns to obtain a diazonium salt of meta-toluidine sulfate;
hydrolysing the diazonium salt of meta-toluidine by reacting with a premix of water, and toluene in a hydrolysis reactor unit to obtain a crude meta-cresol solution,
transferring the crude meta-cresol solution to a layer separation unit and separating meta-cresol in an organic layer, and
recovering spent acid in a spent acid recovery unit from an aqueous layer of the layer separation unit,
obtaining a pure meta-cresol from an organic layer.

12. The process as claimed in claim 11, wherein the predetermined temperature in the step of obtaining is 5-25° C., and wherein a residence time is maintained within 0.1 minutes to 15 minutes.

13. The process as claimed in claim 11, wherein a predetermined ratio of O₂ and NO and gas is maintained between 1:10 to 1:4.

14. The process as claimed in claim 11, wherein a predetermined temperature of preparing a meta-toluidine sulfate salt is maintained between 15° C., and wherein a molar ratio of Meta-toluidine: 98% sulfuric acid is maintained between 1:6 to 1:3.

15. The process as claimed in claim 11, wherein in the step of passing the gas mixture and the meta-toluidine sulfate salt through a diazotization chamber comprising the set of packed columns having different types of packings selected from structured or random packings.

16. The process as claimed in claim 9, further comprising a step of removing excess NOx gases from diazonium salt of meta-toluidine by incorporating sulfamic acid to the excess NOx removal unit and thereby suppressing a formation of N₂O₄, wherein a predetermined temperature at the step of removing excess NOx gases is between 0-5° C.
